(19) United States
(12) Patent Application Publication
(10) Pub. No.: US 2026/0297812 A1
(43) Pub. Date: Oct. 1, 2026

(54) PRODUCTION OF HYDROGEL FIBRES

(71) Applicant: MOSA MEAT B.V., Maastricht (NL)

(72) Inventors: Eric Wolfgang WIELAND, Maastricht (NL); Tessa Merle VAN HAAFTEN, Maastricht (NL); Amit CHANDRAKAR, Maastricht (NL)

(73) Assignee: MOSA MEAT B.V., Maastricht (NL)

(21) Appl. No.: 19/489,367

(22) PCT Filed: Jun. 3, 2024
(86) PCT No.: PCT/EP2024/065157

§ 371 Date: Dec. 2, 2025

(30) Foreign Application Priority Data
Jun. 5, 2023 (NL) 2034998

(51) Int. Cl. D01D 5/38; C12N 5/00; D01D 5/06; D01D 5/34; D01D 13/00

(52) U.S. Cl. CPC D01D 5/38; C12N 5/0018; D01D 5/06; D01D 5/34; D01D 13/00; C12N 2513/00; D10B 2201/00

(57) ABSTRACT

Modular flow cell for production of co-axially layered hydrogel fibers, comprising a plurality of sections, wherein each of the plurality of sections is in fluid communication with an adjacent section of the plurality of sections, and wherein the plurality of sections comprises: an inlet section; a first fabrication section; a second fabrication section and a cross-linking section; wherein the modular flow cell comprises at least one module, wherein the at least one module comprises at least one section of the plurality of sections.

### TECHNICAL FIELD

The invention is in the field of production of hydrogel fibres, preferably hydrogel fibres appropriate for cell culture, preferably cell cultures for the production of cultivated meat, more preferably co-axially layered hydrogel fibres.

### BACKGROUND OF THE INVENTION

The use of hydrogel fibres for cell cultivation is known, for example from ONOE, HIROAKI et al, Metre-long cell-laden microfibres exhibit tissue morphologies and functions, Nature Materials, Vol. 12 Jun. 2013, published online 31 Mar. 2013. FIG. 1 (b) of Onoe et al. shows a microfluidic assembly appropriate to produce a double layered co-axial hydrogel fibre. ECM (extra cellular matrix) protein laden with cells is first introduced in the central capillary, a sodium alginate solution is fed from a concentric channel surrounding it, after which the ECM protein laden with cells will be surrounded by sodium alginate, and finally a third concentric channel will be used to feed the outer surface of the sodium alginate with a CaCl₂ cross-linking solution, forming a double layered co-axial hydrogel fibre containing cells in the core layer. This type of device is usually used in laboratory research conditions and suffers from several issues that prevent it from being scaled up to industrial manufacturing conditions (as in, bigger quantities, shorter manufacture times, etc.), such as being made from glass, a fragile material, the sodium alginate and cross-linking solutions inlets being mounted on the glass tubes as a separate ringed structure and for example not being adapted to form more than one fibre at once.

A disadvantage of the current state of the art is that it is not possible to the manufacture hydrogel fibres on a large scale, e.g. an industrial or commercially interesting scale, especially for cell cultures in the field of cultivated meat. Large volumes of these kinds of fibres have never before been required, but are foreseen to play a large role in the scale-up of cultivated food technologies.

The invention aims to counteract the above disadvantages, preferably while retaining the advantages. More specifically, the invention aims to provide for a device that allows for the production of a large quantity of hydrogel fibres in a relatively short time.

### SUMMARY OF THE INVENTION

The invention provides for a modular flow cell for production of co-axially layered hydrogel fibres, in particular the modular flow cell according to claim 1, comprising a plurality of sections. Each of the plurality of sections is in fluid communication with an adjacent section of the plurality of sections. The plurality of sections comprises an inlet section arranged to receive at least a first material, a second material and a crosslinking solution via corresponding connectors. The plurality of sections further comprise a first fabrication section arranged to receive the first material from the inlet section and feed it to a second fabrication section via at least two main channels, forming a first layer fluid flow, and receive the second material and crosslinking solution from the inlet section and feed it to a second fabrication section via individual first fabrication section channels. The second fabrication section is arranged to receive the first layer fluid flow and the second material from the first fabrication section, and feed the second material concentrically around the first layer fluid flow, thereby forming a co-axial fluid flow. The second fabrication section is further arranged to feed the co-axial fluid flow to a cross linking section via the at least two main channels and the crosslinking solution via a corresponding second fabrication section channels to the cross-linking section. The cross-linking section is arranged to receive the co-axial fluid flow and crosslinking solution from the second fabrication section, and feed the crosslinking solution concentrically around the co-axial fluid, thereby forming a co-axially layered hydrogel fibre in the at least two main channels. The modular flow cell comprises at least one module, wherein the at least one module comprises at least one section of the plurality of sections.

Providing a modular flow cell with multiple sections, via modules, allows for scalability and customizability. A module may comprise a plurality of the same sections in parallel, e.g. a module may comprise multiple first fabrication sections in parallel, facilitating the production of multiple first layer fluid flows parallel. If the other modules are provided accordingly, parallel production of co-axially layered hydrogel fibres may be allowed. Scalable parallel production of the co-axially layered hydrogel fibres allows for the production of a large quantity of hydrogel fibres in a relatively short time. Furthermore, the modular flow cell provided in this manner is easily adaptable to modifications in the production process. For example modules may be replaced, without necessarily requiring the replacement of other modules of the flow cell. Also, a module may be added or removed anywhere in the modular flow cell, provided that adjacent modules after adding or removal correctly interconnect. In case such proper interconnection cannot be established by such adding or removal, further intermediate modules may be added to provide such interconnection. As a result, the modular flow cell of the present invention is very well suitable for industrialization of the process, is scalable and adaptable to later developments, and may be combined with further modular flow cells in a flow cell array to allow for the production of large quantities of hydrogel fibres in a relatively short time.

In the shown examples, each section is represented by a corresponding module. However, it will be clear to the person skilled in the art that a module can comprise a plurality of sections or that one section is distributed over multiple modules.

The plurality of sections further can comprise a distribution section provided upstream of the first fabrication section arranged to direct the at least first material, second material and crosslinking solution from an upstream section into a at least three distribution section channels. The distribution section channels can have a geometry adapted to connected to a downstream section and are arranged to keep the first material, second material and crosslinking solution separated.

The plurality of sections can further comprise a multiplying section provided downstream of the distribution section. The multiplying section can be arranged to receive each of the first material, second material and crosslinking solution from the distribution section channels in at least one individual multiplying section channel, wherein each of the at least one individual multiplying section channels can divert in to a plurality of multiplying section subchannels arranged to feed the first material, second material and crosslinking solution to a further downstream section. The multiplication section allows for a single modular flow cell to produce multiple hydrogel fibres simultaneously, such as to increase the yield of hydrogel fibres being produced by the flow cell. As may be appreciated, the number of simultaneous fibres that can be produced by a single flow cell is limited by the capacity of it's inlet section, but further increasing the yield of a production arrangement may thereafter be achieved by adding additional modular flow cells in an array of any suitable layout.

The plurality of sections further can comprise a restriction section provided upstream of the first fabrication section arranged to substantially equalize the pressure in the first fabrication section channels via restriction channels. The restriction channels can have a cross-section that is smaller than the upstream channels.

The plurality of sections further can comprise a merging section provided downstream of the cross-linking section arranged to merge multiple co-axially layered hydrogel fibres. The merging section can comprise a plurality of merging section subchannels arranged to receive a single upstream formed co-axially layered hydrogel fibre per subchannel. The multiple subchannels can merge in to a merging section channel downstream of the merging section subchannels.

The co-axially layered hydrogel microfibres can be appropriate for a cell culture, preferably appropriate for cultured meat applications.

The at least one module is provided by a plurality of modules, wherein each module of the plurality of modules is configured for being fixed to at least one further module of the plurality of modules, for enabling the plurality of modules to jointly provide the plurality of sections.

The plurality of modules can comprise at least one element of a group comprising: an inlet module for providing at least the inlet section, a first fabrication module for providing at least the first fabrication section, a second fabrication module for providing at least the second fabrication section, a cross-linking module for providing at least the cross-linking section.

The plurality of modules can comprise at least one third fabrication section. The at least one third fabrication section can be configured for receiving the co-axial fluid flow and at least one further second material from an upstream module, for feeding the further second material concentrically around the co-axial fluid flow for adding an additional co-axial layer thereto, and for feeding the co-axial fluid flow to a downstream module via the at least two main channels. As may be appreciated, even further fabrication sections may be added in this manner to potentially provide a fourth, fifth, M-th fabrication section (where M in principle equals any desired natural number).

The at least two main channels can be provided by a plurality of N main channels for the simultaneous production of N co-axially layered hydrogel microfibres in an industrial production arrangement.

The invention further provides for a use of a modular flow cell, preferably the modular flow cell previously described, to cultivate a cell culture comprising one or more animal cells isolated from their original living organism.

The invention additionally provides for a transport system arranged to transport co-axially layered hydrogel fibres. The transport system comprises at least one transport tube arranged to transport co-axially layered hydrogel fibres. The transport system further comprises at least one hydrogel microfibre inlet to the at least one transport tube, preferably a hydrogel microfibre inlet arranged to feed co-axially layered hydrogel fibres produced by the modular flow cell according to any of the preceding claims to the at least one transport tube. Additionally, the transport system further comprises at least one outlet arranged to feed the co-axially layered hydrogel fibres from the transport tube to an auxiliary storage means.

The transport system can further comprise diverting means arranged to direct the co-axially layered hydrogel fibres in the transport system.

The invention further provides for a module for use in a modular flow cell as previously described.

The module can be at least one element of a group comprising: an inlet module for providing at least the inlet section, a first fabrication module for providing at least the first fabrication section, a second fabrication module for providing at least the second fabrication section, a cross-linking module for providing at least the cross-linking section.

### DESCRIPTION OF THE DRAWINGS

FIG. 1—Flowcell for fibre fabrication according to an embodiment of the invention for making sixteen fibres simultaneously.

FIG. 2—Exploded view of the flowcell from FIG. 1.

FIG. 3—An inlet module.

FIG. 4—A distribution module.

FIG. 5—A multiplying module.

FIG. 6—A restriction module.

FIG. 7—A fabrication module.

FIG. 8—A further fabrication module.

FIG. 9—A dual cross-linking/outlet modules according to an embodiment of the invention.

FIG. 10—A section view of the modules of FIGS. 7, 8 and 9.

FIG. 11—Flowcell with merging section for fibre fabrication according to an embodiment of the invention for making sixteen fibres simultaneously and outputting them through a single nozzle.

FIG. 12—An alternative cross-linking module according to an alternative embodiment of the invention.

FIG. 13—A merging module.

FIG. 14—A dual merging/outlet modules.

FIG. 15—Full view of the liquid flow paths of the embodiment of FIG. 11, or the liquid flow paths of the embodiment of FIG. 1 with an added merging section.

FIG. 16—Detail of the moment of fibre crosslinking with velocity profile.

### DETAILED DESCRIPTION OF THE INVENTION

“Fibre”, as used herein, refers to a string-like shaped hydrogel object with a cross section of a circular or mostly circular shape, with a diameter in the order of hundreds of microns, depending on the flow cell. For example, the core of the fibre may have a diameter in the order of 200-400 microns. Said core may have one additional layer, for a total diameter in the order of 800 microns. However, variations will be possible in which the fibre has a diameter in an order of magnitude of 1 mm. A full flow cell may produce fibres of a wide range of diameters, however it is currently expected that for cultured meat the size will be not much larger than 3 mm.

“Co-axial”, as used herein, refers to the disposition of the layers in a multilayer fibre. When a section transversal to the length of the fibre is taken, the several layers will show in a concentric fashion, from the core layer (the innermost) to the outer layer (the outermost). In this document, the core, most inner, layer may be referred to as the “first layer”, and further layers, from the core to the outside of the fibre, as second, third, etc. “Co-axial” will be used to mean “concentric” when referring to the fibres. The word “concentric” will mostly be reserved for descriptions of the nozzles that produce the fibres. Or, in other words, what is meant is that for ease of understanding, even if both the fibres and nozzles can be seen as at the same time being described by the words co-axial and concentric, it is preferred to describe the fibres as co-axial due to their elongated shape (as an analogy to co-axial electrical cables) and the nozzles as concentric to stress the nature of their physical make up, with concentric outlets.

The fibres in this invention comprise at least an outer layer of a cross-linked hydrogel, and may comprise further inner layers of hydrogel and/or other materials, such as extra-cellular matrices like collagen, fibronectin, vitronectin or other ECM proteins.

### Hydrogels as Cell Culture Scaffold

In the biotechnology technical area, certain biocompatible hydrogels have long been known to be appropriate to use as scaffolds for cell cultures, especially for those cell types that require a physical surface to attach to, usually called adherent cells. Certain non-adherent cells may also benefit, depending on the culture conditions, from the presence of a scaffold, for example by being encapsulated by it, thus diminishing their mobility. A scaffold can also be an integral part of a tissue engineering application requiring maintaining a certain spatial positioning of cells, or as a way to prevent cell anoikis (programmed cell death by detachment from the correct type of extracellular matrix). The definition of scaffold is somewhat fluid in the literature, but, in general, a scaffold may be taken to be a structure that either encapsulates the cells, thus mostly immobilising them, or serves as a surface for cells to adhere to. In the context of this invention, a scaffold is to mean a support structure for the cells, independent of whether they are encapsulated in it or at its surface, chemically bound to it (for example through the extracellular matrix of a cell) or not. In short, a scaffold may provide a substrate for cell attachment, cell proliferation, differentiation, and cell migration. It is sufficient that the cells are in contact with the described fibres for them to be defined as a scaffold. A cell culture microcarrier or carrier is also defined as being a scaffold for the purposes of this invention.

It is not an item in this application to delve deep into biological issues, since it strictly concerns production of fibre scaffolds, but some discussion of these will at times be necessary. Cell cultures are well known in the common general knowledge of the biotechnology area, so little discussion of these will be included in this specification, except as follows: a cell culture, as referred to herein, is to mean one or more animal cells, isolated from their original living organism (the animal), subject to artificial conditions that replicate the biological processes that allow them to proliferate or differentiate. These artificial conditions may comprise forming a scaffold for supporting the cells and placing the cells and scaffold in an appropriate bioreactor, petri dish or any other industrial or laboratory equipment adequate for the culture of cells. During culture, the cells and scaffold are mostly surrounded by, or immersed in, a cultivation medium appropriate for culturing the specific cell type. This medium can be specifically tailored to promote proliferation, differentiation, or both. The scaffold is preferably made from a material that allows permeation by the medium, such as a hydrogel.

Cell cultures may be used to produce cultivated meat, an emerging subfield of biotechnology where the meat of an animal, especially the muscle component, is a product of a cell culture process and not processed from an actual living animal, for example in a slaughterhouse/butcher shop. Nowadays, the cultivated meat field is becoming well known and requires little explanation. These techniques are sometimes synonymously referred to as “cellular agriculture”, “lab grown meat” or other terms.

The animal may be any of those mammals usually consumed by humans, such as bovines, ovines or porcines, but is preferably a bovine.

The cells described in this invention are, in a non-limiting fashion, preferably for human consumption, more preferably primary cells, more preferably bovine primary cells, more preferably Satellite Cells (SC's), also known as myosatellite cells. Other well-known cell types for production of cultivated meat are Fibro Adipogenic Precursors (henceforth FAP's) or any type of stem cells, either pluri or multipotent, or Induced Pluripotent Stem Cells (iPSCs). Both FAP's and SC's have been extensively studied and their definition is well known both in the general biotechnology area and the particular cultivated meat area. For the avoidance of doubt, a short explanation of these types of cells follows:

FAP cells are known to support the activation and differentiation of muscle satellite cells and also for having the ability to differentiate into adipocytes. This adipocyte formation mechanism is widely regarded as a way to produce edible fat for cultivated meat applications. FAP's can be found in muscle tissue.

SC's are multipotent cells found in mature muscle and serve as precursors to skeletal muscle cells. SC's may contribute further satellite cells to the muscle tissue or may differentiate into skeletal muscle cells. SC's are known to be involved in muscle reparation processes.

The cultivated meat field has made extensive use of SC's in trying to obtain an edible cultivated meat product, also using cultivated fat (FAP's or other fat cells) to provide taste to the muscle portion of the final food. Any other type of cells used in cultured meat can be part of the invention such as mesenchymal stem cells, adult stem cells, pluripotent stem cells, myocytes, adipocytes, chondrocytes, or fibroblasts, for example.

### Hydrogel Fibres

A vital issue with cell cultures, common to any type of culture vessel, scaffold material or their shape, is the ability to deliver oxygen and nutrients to the cells. An inappropriate delivery of both or any of these two components may lead to poor culture performance or even cell death. Many scaffold shapes have been trialled and typical ones include spheres and fibres. Cell containing hydrogel fibres, in particular, are appropriate for cell culture due to their innate physical shape: the long fibres tend to curl up, filling the available culture volume, but do not form bends beyond a certain angle determined by their rigidity and the overall culture conditions. This creates free space between the fibres wherein culture medium can circulate, providing the required nutrients to the cells. The hydrogel material is by its nature easily permeated by the culture medium, allowing for efficient chemical exchanges from the medium to the cells. Especially for cultivated meat applications, large volumes of cells need to be produced, so a process to manufacture these hydrogel fibres at industrial scales, as disclosed in this invention, is desired.

For some cell types, especially muscle precursors like Satellite Cells, the physical placement of the cells is quite important. Muscle cells tend to differentiate in fibre-like shapes, forming elongated muscle fibres. A scaffold shape that naturally supports the preferred structural shape the cells will organise themselves in is thus especially advantageous over others. In the cultivated meat field, long muscle fibres are able to better replicate the structure of animal meat, when compared to shorter ones, so there is a need to produce longer (and thus more massive) hydrogel fibres.

Hydrogels are usually formed by cross-linking an hydrogel precursor present in a solution. The cross-linking agent is usually also in solution, and by mixing the hydrogel precursor solution with the cross-linking solution, a gel network forms, containing high quantities of water (the solvent), usually above 10%. It is possible that the amount of water goes above 90%, or even 99%.

The flowcell of the invention is compatible with any hydrogel precursor solution, but for the cell culture, and specifically cultured meat for which it's use is preferred, the use of biocompatible hydrogels is preferred. These are usually hydrogels prepared from natural polymers, such as hyaluronic acid, chitosan, heparin, alginate, gelatine, or fibrin, amongst others. The invention is particularly suited for production of hydrogels by cross-linking, of which alginate (for example sodium alginate) is a preferred material. Alginate can be cross-linked by a solution of calcium chloride salt (CaCl₂).

The alginate or other hydrogel precursor may be chemically modified, such as being functionalized in any way. The outer layer of a fibre according to this invention is always of a cross-linkable hydrogel precursor, preferably alginate, but the inner layers may be made up from alternative materials or mixture of hydrogels precursors with other materials. Low molecular weight hydrogel is preferred to support cell growth and high molecular weight alginate is preferred to provide mechanical support to the fibre.

### Flowcell for Hydrogel Fibre Production

When confronted with the problem of how to scale up the production of hydrogel fibres to industrial scale, the inventors devised a solution based on a modular flowcell. Such a modular flow cell comprises a plurality of sections, wherein each of the plurality of sections is in fluid communication with an adjacent section of the plurality of sections. The plurality of sections comprise:
an inlet section arranged to receive at least a first material, a second material and a crosslinking solution via corresponding connectors;
a first fabrication section arranged to receive the first material from the inlet section and feed it to a second fabrication section via at least two main channels, forming a first layer fluid flow, and receive the second material and crosslinking solution from the inlet section and feed it to a second fabrication section via individual first fabrication section channels;
wherein the second fabrication section is arranged to receive the first layer fluid flow and the second material from the first fabrication section, and feed the second material concentrically around the first layer fluid flow, thereby forming a co-axial fluid flow, and wherein the second fabrication section is further arranged to feed the co-axial fluid flow to a cross linking section via the at least two main channels and the crosslinking solution via corresponding second fabrication section channels to a cross-linking section.

The cross-linking section is arranged to receive the co-axial fluid flow and crosslinking solution from the second fabrication section, and feed the crosslinking solution concentrically around the co-axial fluid, thereby forming a co-axially layered hydrogel fiber in the at least two main channels. The modular flow cell comprises at least one module, wherein the at least one module comprises at least one section of the plurality of sections.

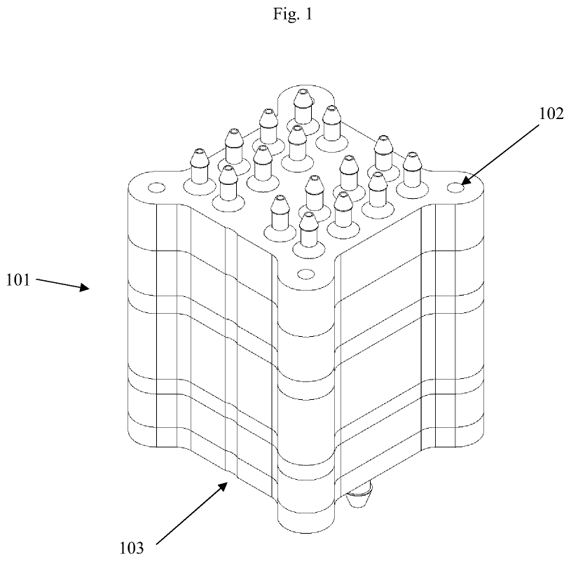

This flowcell is built up from several sections provided in parts, or modules, which are assembled in a certain order, and secured in place by fastening means. The flowcell is then defined as a volume contained between and including two outermost modules, on opposite sides of the flow cell from each other. FIG. 1 may be used to understand what is meant by this, where the modular construction of the flowcell is readily apparent and the disposition of the two outermost modules can be seen. After assembled from the chosen modules, the flowcell can then be connected by means of at least one module configured to allow liquid connections (henceforth known as an “inlet module”), to at least one source of hydrogel precursor fluid, preferably one source being laden with cells, and at least one source of a hydrogel crosslinking solution. Both fluids are then channelled throughout the flowcell according to pathways defined by the modules present between the or several inlet modules and the outlet module or modules. Arbitrarily, the bottom of FIG. 1, where the main fluids are fed to the flowcell will be called the bottom of the flowcell, and the top of FIG. 1, where the hydrogel fibre(s) exit the cell, will be called the top of the flowcell. Each individual module will also have a top base, facing the direction of the top of the flowcell and a bottom base, facing the direction of the bottom of the flowcell. These directions are used only to aid in the description of the invention, but in no way constrain the functioning of the invention. The flowcell may be put in use during actual operation in any required orientation.

### Description on the Overall Geometry of the Modules and Technical Terms Used

The modules comprising sections, either within themselves or between one another may present both with microfluidic and non-microfluidic regions. The non-microfluidic section is called macrofluidic. In general, parts of the flowcells work in the macrofluidic regime and other parts in the microfluidic regime-the flowcell may be said to be a mixed state flowcell. For example, the first few modules may be macrofluidic and at a certain point the modules will provide for a microfluidic regime to allow for the desired effects in fabrication, as the channel dimensions become smaller. An intermediate state, milifluidic, is sometimes used in the literature, which would mean dimensions in the order of milimetres. Certain sections could thus be labelled as working in the milifluidic regime. This will be made clear as the invention is described.

For brevity and avoidance of constant repetition, the “microfluidic” and “macrofluidic” terms will be mostly omitted in this specification. Microfluidic is a well-defined term in the literature and means that several and any dimensions of the channels within a module may be smaller than 1 mm. For example, a channel with a diameter of 700 μm is a microfluidic channel. Eventually, channels may have several and any dimensions above 1 mm, in which case they are not microfluidic in the literal sense of the term, even if potentially approaching this state of functioning. Milifluidic is a term sometimes used in the literature to describe these intermediate situations, Channels or modules may also be clearly fully microfluidic or macrofluidic.

The modules of the invention are shaped as cuboids, preferably as rectangular prisms, square prisms, or cubes. A rectangular prism will have 6 faces where none are squares (all rectangles), a square prism will have two opposite square faces with the remaining 4 being rectangles and a cube will have 6 square faces. The modules may also present as any type of polyhedric prism, for example hexagonal prisms, but simpler forms (and cheaper) to manufacture are preferred. The modules present with internal hollow fluid transport channels, or holes, and usually with orifices or channels on their surfaces. Two diametrically opposed surfaces of the cuboid are the “bases”. These bases are the preferred surfaces where the internal fluid transport channels have their opening(s) to the outside of the module and also the surfaces by which one module is positioned facing another. FIG. 3 for example shows two of the bases of a module with visible openings. The surfaces of the modules that are not bases are called “walls”. The walls are preferably devoid of external openings to the internal fluid transport channels, but may in some cases present with them. The cuboids may deviate from the cuboid shape, particularly the bases, when being provided with a through orifice for the insertion of fastening means, for example a threaded bolt. In this case, the bases may show a bulge around the general area where the through orifice 102 is located. The bases may also be unmodified in their geometry and the through orifices may simply be formed on their original square or rectangular shape. The through orifices for the fastening means will be referred to as fastening apertures. The fastening means may also be external to the modules and no fastening apertures will be required. This is described under its own section (Fastening Means). The modules may also present with slots or similar structures for insertion of gaskets, O-rings or similar parts.

In general, a module will then have: two bases (one a top base and the other a bottom base), preferably 4 but optionally more walls, internal fluid transport channels, sometimes called trough-channels or through-holes, orifices or channels on the surfaces, optionally fastening apertures, optionally bulges around the fastening apertures.

### Manufacture and Materials of Modules

In the shown examples, modules are manufactured by forming channels and/or orifices on either base, typically by subtractive manufacturing (machining) from a solid block of material, and connecting the channels from one face to the other with holes or through-channels. This way, liquid is diverted in specific routes on each base and flow is allowed from one to the other. Eventually, modules can be manufactured by additive manufacturing (3D printing for example), which allows for internal routing of channels that may be mostly impossible to obtain by subtractive manufacturing due to access constraints for machining tools when the channel geometry obscures a direct line of access.

A preferred material for modules is stainless steel, but any other alloys or metals may be appropriate to use-for example, copper or brass may be used. Polymer or ceramic based materials may also be used, but constraints such as ease of sterilisation, mechanical resistance (mostly to compression) and chemical resistance (due to the solutions used) are all to be considered when selecting materials. The module material should preferably not leach chemicals into the fibres or allow particles to detach from the surface of the channels when liquid is flowing through them.

### Types of Modules

The modules can serve several different functions via various sections. The sections of modules are inlet sections, outlet sections, distribution sections, multiplying sections, restriction sections, fabrication sections and cross-linking sections. Other types such as merging sections are possible. In the shown example below, each module comprises a single section. However it will be clear that modules may have more than a single sections, i.e., be dual function modules. In other words, the modular flow cell comprises a plurality of sections, wherein each of the plurality of sections is in fluid communication with an adjacent section of the plurality of sections. The modular flow cell comprises at least one module, wherein the at least one module comprises at least one section of the plurality of sections.

In the shown examples, the inlet modules comprise an inlet section. The inlet section is arranged to receive at least a first material, a second material and a crosslinking solution via corresponding connectors. The inlet modules are modules that present with connectors for feeding liquids in one base, eventually on a wall, and allow these to flow to another module in contact with a base of an inlet module that does not present with connectors. The connectors may accept a plastic hose for example, or any other type of liquid transport device. Considering the geometry of an entire flowcell, as seen in FIG. 1, no matter how many inlet modules are present, only one, the bottom one, will have a base available for connectors. If more than one module are to be inlet modules, then the connectors of the ones not on the bottom of the flowcell must obligatorily have the connectors on the walls.

The number of connectors corresponds to the number of different materials in a hydrogel fibre plus 1, since there must always be at least one hydrogel precursor making up one layer and a crosslinking agent in solution. So, for example, making a two layer fibre would require a flowcell with 2+1 connectors, two for the different hydrogel precursors (or alternatively an hydrogel precursor and another material) of each layer and one for the crosslinking solution. It is in practice possible to make a fibre with two layers of the exact same material, meaning that only one type of hydrogel precursor would be needed, so the flow would have to be managed to divide the path of the single hydrogel precursor solution towards the desired number of layers. Ideally, these types of single material fibres would actually be manufactured as a single layer fibre, avoiding the need for complicated flow paths and this invention.

In the shown example, the distribution modules comprises a distribution section provided upstream of the first fabrication section arranged to direct the at least first material, second material and crosslinking solution from an upstream section into a at least three distribution section channels, said distribution section channels having a geometry adapted to connected to a downstream section and are arranged to keep the first material, second material and crosslinking solution separated. Distribution modules are modules that direct the flow either from the inlet module or a previous dividing module into a channel with a geometry adapted to connect to a multiplying module. For example, a distribution module such as the one in FIG. 4 receives three liquids from its lower base and directs their flow into three longer channels on its top surface. These longer channels are positioned, dimensioned or otherwise adapted to connect to a follow-up multiplying module. The distribution modules are optional. For example, the inlet module, such as the one in FIG. 3, could have a top base already adapted to the shape of the top face of the distribution module. Distribution modules are useful to prevent having other modules locked into certain configurations. For example, with a generic inlet model like the one in FIG. 1, several types of distribution modules could be used, with different channel configurations. This allows the inlet modules to be simpler and easily used in flow cells of varying configurations. The same line of argument concerning the inlet modules is valid for any other type of module preceding a distribution module.

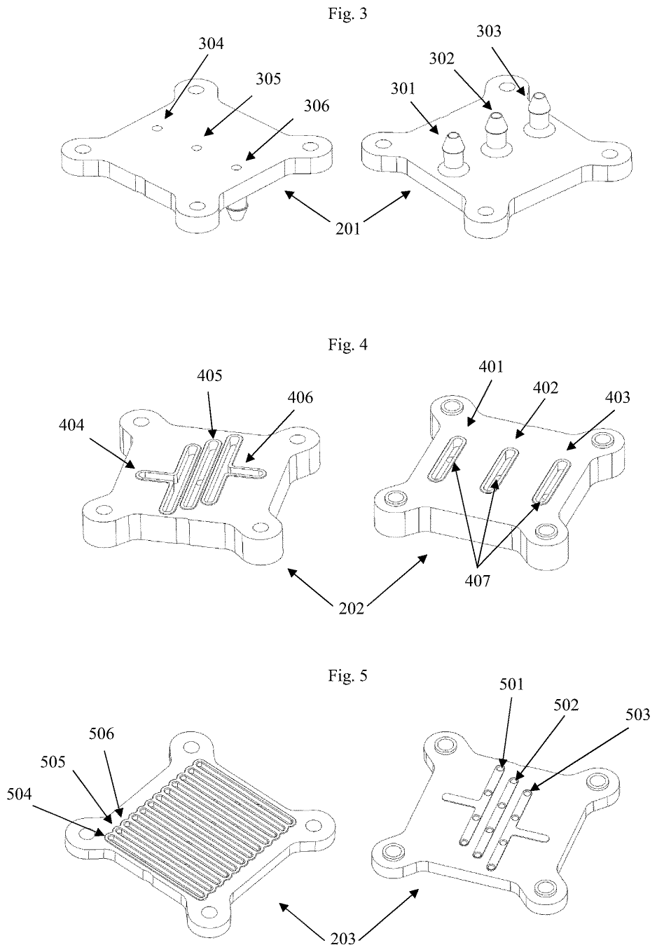

In the shown example, multiplying modules comprise a multiplying section provided downstream of the distribution section. The multiplying section is arranged to receive each of the first material, second material and crosslinking solution from the distribution section channels in at least one individual multiplying section channel, wherein each of the at least one individual multiplying section channels diverts in to a plurality of multiplying section subchannels arranged to feed the first material, second material and crosslinking solution to a further downstream section multiplying section. Multiplying modules are modules that multiply a flow into several outputs. For example, a multiplying module may receive three liquids, each on a single channel, on the lower base and divert these into four channels per liquid type on its upper base. This would be a multiplier factor of 4 per channel in the multiplying module. A module of this type is shown in FIG. 5. Multiplying modules according to the invention may have any multiplying power as desired, by adjusting channel sizes, paths and module dimensions. Obviously, a physically larger base allows for a larger multiplier factor. Also, the number of liquids/solutions fed to the module may vary. Usually, a multiplying module may connect to a fabrication module, another multiplying module, a restriction module or a distribution module. For example, when connected to a fabrication module a multiplying module feeds the former for actual fabrication of fibres; when connected to another multiplying module, further division of the flows may happen, such as a 3 liquid type 4× multiplying module feeding a 2× multiplying module, ultimately forming 3 time 4 times 2, 24 channels per liquid type; when connected to a restriction module the multiplying module simply feeds the former; when connected to a distribution module, the flow may be re-oriented into more appropriate directions, depending on the configuration of the flowcell.

According to the invention, multiplying modules are enabled in any multiplier factor and number of liquid flows, and some examples are given here for clarification of what is meant: for example, a channel with two single channel fluid inputs may transform these into a four channel output per fluid. This would be a 4× factor, single channel 2 fluids module, with a total of 8 outputs. A 8× factor, single channel 4 fluids module would output 4 fluids in 8 channels each, so a total of 32 outputs. A module receiving 3 fluids that have previously been divided could for example be a 2× factor module, triple channel 2 fluids configuration (each type of fluid runs in three channels), this would mean that each of the 3 channels per fluid become 3×2, 6 channels, for a total of 3×6, 18 channels per fluid, and two types of fluid give 2×18, 36 total channels for all fluids.

In the shown example, restriction modules are provided. Said restriction modules comprise restriction sections provided upstream of the first fabrication section arranged to substantially equalize the pressure in the first fabrication section channels via restriction channels. The restriction channels have a cross-section that is relatively small compared to the upstream channels. Restriction modules are optional but definitely preferred modules that serve to help equalize the fluid pressure over all the channels for a certain fluid flow before fabrication starts. Considering all the paths that the fluids travel from the inlet module, eventually through multiplying and distribution modules, depending on the configuration of the particular flowcell, the pressures of the separate fluid flows for the same fluid may start diverging, from channel to channel. Controlling the pressure and fluid flow volumes is important to manufacture good quality, similar and replicable fibres. For example, two channels transporting the same fluid, one with a much higher pressure than the other, may give rise to fibres with very different dimensions or even lack of quality in forming the layers or during the crosslinking stage, with too much or too little material being provided. The restriction module thus solves this problem by being provided with thin linear channels, called restriction channels, over a sufficient thickness to allow for pressure and flow correction. Restriction channels help maintain uniform liquid distribution by introducing a restriction or resistance to the flow of liquid that helps equalize the flow rates through different channels of the same liquid flow. By introducing a restriction or narrowing of the channel, the liquid experiences a pressure drop, which reduces the flow rate through that channel. If multiple channels have restriction holes of the same size and shape o, the liquid will experience a pressure drop resulting in a similar final pressure across each channel, resulting in a more uniform flow rate. Additionally, the resistance created by the restriction channels helps to dampen any variations in the flow rate due to undesired changes in pressure or other factors. Furthermore, by carefully designing and optimizing the dimensions of the restriction channels, it is possible to achieve the desired flow rate through each channel and ensure uniform distribution of the liquid flow, per different flow. Obviously, if one of the liquid flows enters the input module with a different pressure from another one, the final optimized pressure will still be different between liquids. The correct dimensions of the channels are mostly obtained empirically, but it has been found that a good combination of diameter and length is one that prevents clogging of the channels, allows for uniform pressure drops (too narrow channels may cause an excessive pressure drop, and too wide may cause the pressure distribution to diverge from uniformity). An additional consideration when designing the diameter of the channels may be that smaller channels, for example having a size smaller than 1 mm, such as 0.7 mm or more specifically smaller than 0.5 mm, clog and thereby blocking the flow in the channel. The restriction module usually presents with more through channels than the number of channels on the top base of the previous module. In a sense, the restriction module performs the final multiplication of the flows, but differs from a pure multiplying module by having the exact same number of orifices in each base, and no channels at the top base.

A restriction module may provide a significant the total pressure drop on the flowcell, for example a total pressure drop of 30% to 99%, specifically 50 to 90% or more specifically 80%. The through holes in a restriction module may have different diameters for different liquid flows, resulting in different final pressures per liquid.

In the shown example, a first fabrication module and a second fabrication module are provided. Said first fabrication module comprises a first fabrication section arranged to receive the first material from the inlet section and feed it to a second fabrication section via at least two main channels, forming a first layer fluid flow, and receive the second material and crosslinking solution from the inlet section and feed it to a second fabrication section via individual first fabrication section channels. The second fabrication module comprises a second fabrication section arranged to receive the first layer fluid flow and the second material from the first fabrication section, and feed the second material concentrically around the first layer fluid flow, thereby forming a co-axial fluid flow, and wherein the second fabrication section is further arranged to feed the co-axial fluid flow to a cross linking section via the at least two main channels and the crosslinking solution via corresponding second fabrication section channels to a cross-linking section. Fabrication modules are the modules where the fibres are formed. They usually come after a restriction module or a previous fabrication module but can in certain flowcell configurations come after a multiplying or distribution module. Fabrication modules, examples of which can be seen in FIGS. 7 and 8, are made such that the several layers of the fibre are sequentially formed, starting from the inner layer, the core, and moving to the outer layers. As an example, a fabrication module for a two layers fibre would start by having a hydrogel precursor (or another fluid with an alternative material) flow through a channel of circular section that protrudes from the upper base of the channel, i.e., a nozzle. This protruding nozzle is configured to enter an opening on the following module. Through channels will exist to allow the hydrogel precursor fluid for the next layer and the crosslinking solution to flow through the module. The next fabrication module will receive the first layer fluid, and feed the second layer fluid in a concentric fashion with the core flow, forming a co-axial fluid flow. This co-axial flow is also directed to a protruding nozzle, configured to enter the following module, which would in a two-layer fibre case be the cross-linking module.

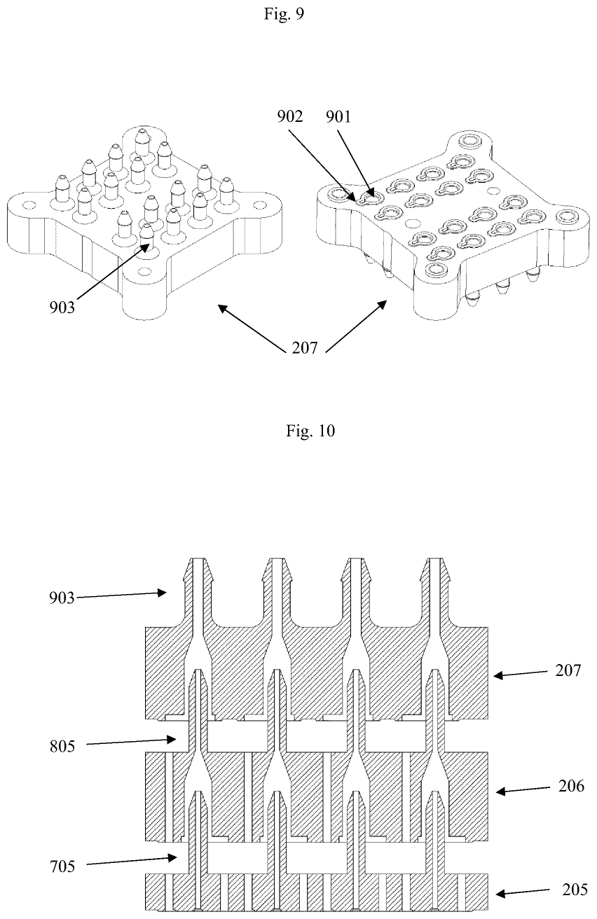

In the shown example, cross-linking modules comprising cross-linking sections are provided. The cross-linking section is arranged to receive the co-axial fluid flow and crosslinking solution from the second fabrication section, and feed the crosslinking solution concentrically around the co-axial fluid, thereby forming a co-axially layered hydrogel fiber in the at least two main channels. Or in other words, in the shown example cross-linking modules are modules that receive the co-axial fluid flow that will form the final cross-linked hydrogel fibre and feed the crosslinking solution to the outer surface of this flow in a concentric fashion. FIG. 9 shows an example of a cross linking module. The, or each, output of the fabrication modules, in the form of a co-axial liquid flow, or a single layer liquid flow in the case of fabrication of single layer fibres, is fed into a channel of a cross-linking module and a flow of crosslinking solution is directed co-axially to this flow by a externally concentric channel to the co-axial liquid flow, or a single layer liquid flow. The co-axial or single layer liquid flow is then cured when in contact with the crosslinking solution and the output of the cross-linking modules are hydrogel fibres, with as many layers as desired.

A further type of modules are merging modules comprising merging sections. Said merging section is provided downstream of the cross-linking section and is arranged to merge multiple co-axially layered hydrogel fibers. The merging section comprises a plurality of merging section subchannels arranged to receive a single upstream formed co-axially layered hydrogel fiber per subchannel. The multiple subchannels merge in to a merging section channel downstream of the merging section subchannels. These modules are optional and serve to merge the formed hydrogel fibres into either a single output or at least less outputs that the final fabrication module. They receive the fibre output of a cross-linking module and merge it, in as many stages as necessary, into bundles of fibres. For example, four nozzles or orifices of a crosslinking module may each respectively feed a single fabricated fibre into four orifices of the base of a merging module. This quadruple flow may then be combined by having merging channels and leave the top base of the modules through a single orifice or nozzle. The outputted flow now transports four fibres at once.

Outlet modules are usually dual function sections, i.e. modules with two sections. These are the modules topmost on the flowcell. After these modules, the flow of fluids and/or fabricated hydrogel fibres proceeds beyond the confines of the flowcell. A cross-linking module may have a dual function as an outlet modules. A merging module may also have a dual function as an outlet modules.

If necessary, any module, but preferably the inlet module or a module close to the inlet, may present with standard backflow valves, such as Duckbill Valves from mini Valve, for example the ones with ref. DU 027.002 SD. Backflow valves impede the liquids from flowing back through the wrong channels especially in areas of the flowcell where the pressure is not equalized. For example, flowing of the cross-linking solution into channels transporting hydrogel precursor may cause premature cross-linking and possibly clogging issues may arise. In a special embodiment, a flowcell may be provided that does not comprise any backflow valves, in particular when the flowcell is designed such that the pressure is equalized throughout all the fluids.

### Fastening Means

In the shown example, the modular flow cell comprises a plurality of modules. Each module of the plurality of modules is configured for being fixed to at least one further module of the plurality of modules, and for enabling the plurality of modules to jointly provide the plurality of sections. After assembly of the required modules in the required order to form a flowcell, the modules may be closed together by inserting a threaded screw or similar in the fastening through holes and tightening the modules by using a nut for example, or clamping the modules together in a tightening cradle. The tightening cradle may be formed by any structure that holds the modules in order and may be tightened in any way. The cradle option is actually preferred since this allows the modules' base areas to be as small as possible, without the need to use any space for fastening means on the modules themselves.

### Leakage Prevention

To prevent leakage between modules, typical solution used in fluid applications are used: gaskets may be formed that correspond to the orifices or channels on the several modules, indentations or similar may be formed for O-rings or other similar solution and protruding parts on the surfaces of the bases of the modules (such as seen, exemplarily, in the drawings of modules) may be used to help conform the geometry to the gaskets or O-rings or similar means, helping to increase the closure force where necessary and prevent leakage.

### Alginate Hydrogels

Various commonly known hydrogels can be used in the flowcell, such as for example alginates. Alginates, especially sodium alginate, are composed by two monomers in different rations and orders. These are β-D-mannuronate (M) and α-L-guluronate (G). The physical properties of alginate hydrogel are closely related to its M/G ratio.

Alginate is thus the whole family of linear copolymers containing blocks of M and G residues. The blocks are composed of consecutive G residues (GGGGGG), consecutive M residues (MMMMMM), and alternating M and G residues (GMGMGM). Extensive literature exists on how these residues can be organized to achieve a final hydrogel network with different mechanical properties, including higher and lower stiffness or tensile strength. The molecular weight of the final copolymer is also a good generic indicator of the mechanical properties of the hydrogel, with higher molecular weights generally corresponding to a higher stiffness and/or tensile strength. The manufacture or provision of alginate hydrogel precursors is not a concern of this invention since the person skilled in the art is aware of and can pick from several commercial compositions that are available in the market and that can be combined in the flowcell of the present invention to achieve fibres with layers of certain mechanical properties.

### Example of General Operation of a Flowcell

In operation, a flowcell will be connected to at least one source of hydrogel precursor solution, preferably at least two, and a source of cross-linking solution, for example by connecting transport hoses to an appropriate connection on the bottom base of an inlet module. These at least two flows (cross-linking fluid and at least one hydrogel precursor fluid) leave the inlet module and proceed into a distribution module, where they are then diverted into channels on the top face of the distribution module with an appropriate geometrical shape to allow feeding each flow to multiple orifices in the bottom base of a multiplying module. The multiplying module presents with several orifices distributed along the path of each channel of the distribution module and diverts the fluid of the distribution module into several channels on the multiplying module's top base. The initial flows may now have been multiplied to several channels. Another distribution module or multiplying module may now be present, depending on the configuration of the flowcell according to the desired number of fibres and number of layers.

Usually after a multiplying module a restriction module now receives the multiplied flows, having the total number of orifices corresponding to the number of fibres to fabricate times the number of layers desired. The liquids flow linearly through the restriction module and a pressure drop happens that equalizes the pressure throughout all the through channels at the time the flows reach the top base of the restriction module.

The restriction module then feeds into the first of as many fabrication modules as necessary to form the fibres, from the innermost to the outermost layers and finally a cross-linking module creates the hydrogel network and effectively finishes the fabrication of the fibres. The cross linking module may serve as an outlet modules, meaning that the fibres will leave the flowcell at that point and proceed to whatever final destination is required, usually a further transport system or to accumulate into a vessel, or the cross-linking module may feed the fibres into a merging section, where eventually the fibres are combined into one or several bundles of fibres with the desired amount of merged fibres per bundle.

When it comes to manipulating the final dimensions of the fibres, and without being limited by theory, the inventors believe that one of the most important issues is maintaining a laminar flow between the layer fluids and the crosslinking solution in at the moment of crosslinking. As long as this laminar flow is maintained, a final fibre with well defined layers will be produced. In terms of varying the thickness of the several layers, a combination of input pressure for each fluid and dimensions of the nozzles can be used to control how each of the fluid flows thins or thickens as the fluids are combined. It is generally better to simulate the flowcell with Computation Fluid Dynamics (CFD) to determine the ideal conditions for obtaining a certain set of fibre dimensions. Of course, this determination can be done empirically, by trial and error, for example increasing the pressure and thus the flow of a fluid corresponding to a layer that one wishes to have thicker. In FIG. 16 a detail of a specific case may be seen: a core flow—1603 and a sheathe flow 1602 exit a nozzle 805 in a laminar flow mode and are subject to the pressure of the cross-linking solution 1601, causing the total diameter of the flow to become smaller. Cross-linking is known to happen in microseconds, so, before the laminar flow is disrupted, a hydrogel fibre is formed. In terms of average velocity profile, superposed at the top of the figure under reference 1604, one can see that the central section that becomes the fibre is compressed and accelerates, traveling faster than the outer section of the flow, corresponding to the cross-linking solution. This can be adjusted so that for example, with a lower pressure of the cross-linking solution, a thicker fibre is formed. The same concept applies to the moment when a core precursor liquid flow contacts the liquid flow of a further layer, with the combination of flow rate/pressure/channel dimensions contributing to how thick both layers become. Again, the inventors stress that a good CFD simulation can be invaluable when trying to obtain certain fibre dimensions.

### Hydrogel Fibre Bundle Transport System

In an alternative embodiment, the invention also relates to a transport system for fibre bundles. The transport system is arranged to transport co-axially layered hydrogel fibers and comprises:
at least one transport tube arranged to transport co-axially layered hydrogel fibers;
at least one hydrogel microfiber inlet to the at least one transport tube, preferably a hydrogel microfiber inlet arranged to feed co-axially layered hydrogel fibers produced by the modular flow cell according to any of the preceding claims to the at least one transport tube;
at least one outlet arranged to feed the co-axially layered hydrogel fibers from the transport tube to an auxiliary storage means.

The transport system may comprise diverting means arranged to direct the co-axially layered hydrogel fibers in the transport system. After hydrogel fibres are manufactured and merged into bundles, these bundles may need transporting, for example through a switchable fluid circuit that may feed several vessels, such as bioreactors. For example, when one vessel is full, the fabrication section of hydrogel fibres may work to fill a different vessel, preventing having extended downtime on the fabrication equipment, and reducing the amount of fabrication equipment necessary.

The inventors propose a schema where the fluid on which the bundles are transported after fabrication is transferred to a dedicated transport system.

In an industrial installation where several bioreactors are being used for cell culture on hydrogel fibres, using for example the cell types of this specification, and also for example for cultured meat applications also as described in this specification, it may be advantageous to be able to separate the hydrogel manufacture unit from a specific bioreactor. Ideally, a fabrication site for fibres could for example include several units to produce fibres, such a bank of the flowcells described herein, and the output of each of these flow cells could be transported to select locations, optionally allowing for merging the fibres into bundles or already preformed bundles into larger bundles, or with no merging whatsoever, also allowing for any necessary diversions of the flow to change paths as required.

The transport system consists of at least one transport tube, at least one hydrogel fibre inlet, an outlet and optional diversion means.

As an example, a specific transport system is described:
A bank of hydrogel fibre fabrication devices is provided. The fabrication devices may for example be the flowcells of this specification. Each device outputs individual fibres from individual outputs, or outputs a merged bundle of fibres. Individual fibres or bundles may be combined into a single flow, formed by the fluid exiting the hydrogel fabrication devices. During operation, the fabrication devices are continuously fed with fluids at certain pressures such as hydrogel precursor fluids and cross-linking fluids, and these fluids contribute to push and transport their output after the fibres leave the fabrication devices. This flow may be called the fabrication flow of the fabrication device. In a flowcell as described in this specification, a fabrication flow will continuously push the fibres or bundles after these exit the flowcell. The initial transport means from the fibre fabrication bank, merged or not, will eventually be led into the transport system. The transport system inlet consists of a tube, with a downstream and a upstream direction, where the upstream direction is the one onto which the fibres are to flow towards. This may be accomplished simply by using the fabrication flow of the fabrication bank, or, preferably, the transport system may have its own fluid running inside, the transport flow, exerting the necessary pressure to correctly orient the fibres upstream. One advantage of providing the transport system with its own transport flow is that the fabrication flow through the fabrication units can eventually be stopped while still maintaining pressure in the transport system. For this, the inlets into the transport system are fitted with valves that when closed will prevent fluid backflow back into an inactive fabrication device. After entering the transport system, the fabrication flow will now be part of the transport flow, so the fabrication flow is no longer referred to.

After the fibres or bundles are fed to the transport system by one or more inlets, the transport flow of transport fluid in the transport system will push the fibres along the tube or tubes of the system. Diverting means may exist, such as closable valves, to direct the transport flow to desired pathways. At a certain point, an outlet of the transport system will be reached whereupon the fibres or bundles will leave the transport system, for example by entering a vessel, such as a bioreactor. The transport flow accompanies the fibres or bundles into the vessel and exits the transport system. This is a zone of great pressure drop and loss of mass for the transport system, and as such the pressure, speed, flow rate and/or volume of transport flow is optimized to compensate for the flow rate of the transport flow exiting the transport system. The transport system is provided with pumps or other appropriate means to maintain the correct flow properties of the transport flow. Alternatively, when the transport system uses exclusively the fabrication flow(s) as transport fluid, the necessary pumping systems will be associated with the fabrication devices.

### Further Exemplary Embodiments of the Invention

The further exemplary embodiments of the invention will also be used to provide a detailed description of some of the figures. This choice stems from the fact that the invention is difficult to put in words and as such the figures are more easily explained in the context of the embodiments, but it should be clear that the specific figures shown are exemplary only and variations are possible. Also, all technical features of all embodiments can be freely combined amongst themselves providing there isn't a clear technical incompatibility between some options. FIGS. 3 to 9 all show a view of the module from the top on the left and on a right a view from the bottom. The views are mostly but not perfectly isometric since small rotations were applied to allow seeing relevant details, such as trough-channels.

### A Flowcell for Simultaneous Fabrication of Sixteen Two-layer Hydrogel Fibres

A non-limiting example of a specific flowcell is now described. This flowcell is configured to fabricate a hydrogel fibre with two layers of hydrogel with different properties, outputting sixteen finished fibres at once. For this, there should be three liquid inputs, two for the hydrogel precursor fluids and one for the cross-linking solution, and sixteen cross-linking channels in a dual cross-linking/outlet modules.

FIG. 1 shows an overall view of a flowcell 101 configured for such a fabrication process. FIG. 1, and most drawings of the modules, shows a version of the modules where all present with four corner fastening means 102, in this case in the shape of bulges with concentric holes for threaded bolts or similar mechanical fastening means. As said before, the fastening means are not limited to this type. Notch 103 is provided in the drawings of all modules to help determine orientation from figure to figure. In actual use, the notch may be present to help orient the modules.

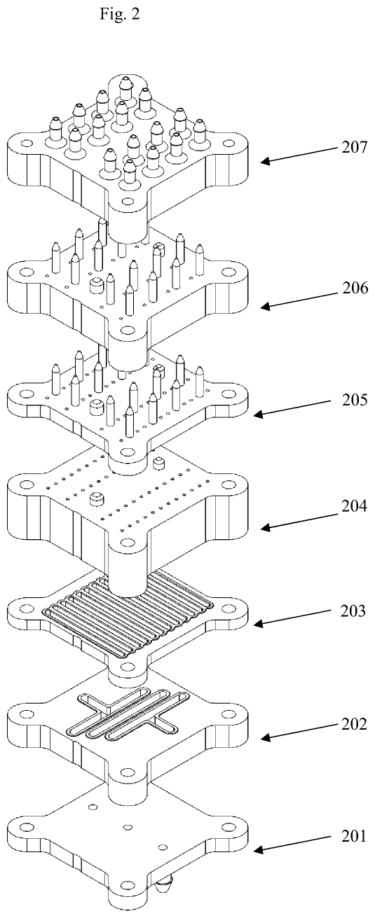

FIG. 2 shows an exploded view of the modules that form part of the flowcell 101 of FIG. 1. From bottom to top, there is an inlet module 201, a distribution module 202, a multiplying module 203, a restriction module 204, two fabrication modules 205 and 206 and a cross-linking module 207. In the embodiment, each module comprises a corresponding section. However, it will be clear to the person skilled in the art that this is a non-limiting example, and that a module can comprise multiple sections.

Three hoses or other normal types of connectors for liquids are connected to the liquid inlets 301, 302, 303 of the inlet module 201 of FIG. 3. 302 is the inlet for the first layer hydrogel precursor fluid (or, core fluid, or fluid A); 303 is the inlet for the second layer hydrogel precursor fluid (or, sheath fluid, or fluid B). 301 is the inlet for the cross-linking solution. For the preferred application of cellular agriculture, both, or alternatively fluid A or fluid B may present with cells in suspension, preferably animal cells. In this example, fluid A presents with undifferentiated bovine satellite cells in suspension. In this example, the hydrogel is sodium alginate, and fluids A and B present with different ratios of M/G. Fluid B, the sheathe fluid, has a precursor for a higher stiffness hydrogel than fluid B. So, fluid B will provide for mechanical stability and fluid A for cell mobility and easier nutrient diffusion in culture condition provided for after manufacture of the fibres.

Inlet 301 allows fluid to flow through the module from the bottom out from top orifice 304, inlet 302 corresponds to orifice 305 and inlet 303 corresponds to orifice 306.

The fluids then flow into a distribution module. FIG. 4 shows a module 202 of this type according to this particular embodiment. Orifice 304 feeds channel 401 (cross-linking solution), orifice 305 feeds channel 402 (fluid A) and orifice 306 feeds channel 403 (fluid B). Channels 401, 402 and 403 present with a through hole 407 each, and feed the top channels 404, 405 and 406, respectively.

Next, a multiplying module 203 is present, as can be seen in FIG. 5. The module's bottom base is configured to block the top channels of the previous module 202 except in select orifices. This is achieved for example with a blind/solid surface area that effectively blocks liquid distribution. This area can be combined with gaskets O-rings or any appropriate similar means to stop leakage. Orifice 501 receives the flow from channel 404, the cross-linking solution. Orifice 502 receives the flow from channel 405, fluid A. Orifice 503 receives the flow from channel 406, fluid B. Three more orifices of each type, seen disposed in a straight line, receive the same fluid flows. These flows will be used for further multiplications of the fluids. We will focus on a first multiplication as an example.

The orifices on the base of the multiplying module 203 serve as through-channels to the top base of the module. On the top base, 12 channels can be seen. Basically, these channels are arranged in groups of three: orifice 501 connects to channel 504, orifice 502 connects to channel 505 and orifice 503 connects to channel 506. This combination is repeated in threes for the following channels, or, as can be seen, a repetition of the structure cross-linking solution, fluid A and fluid B in four times, giving a total of 12 channels.

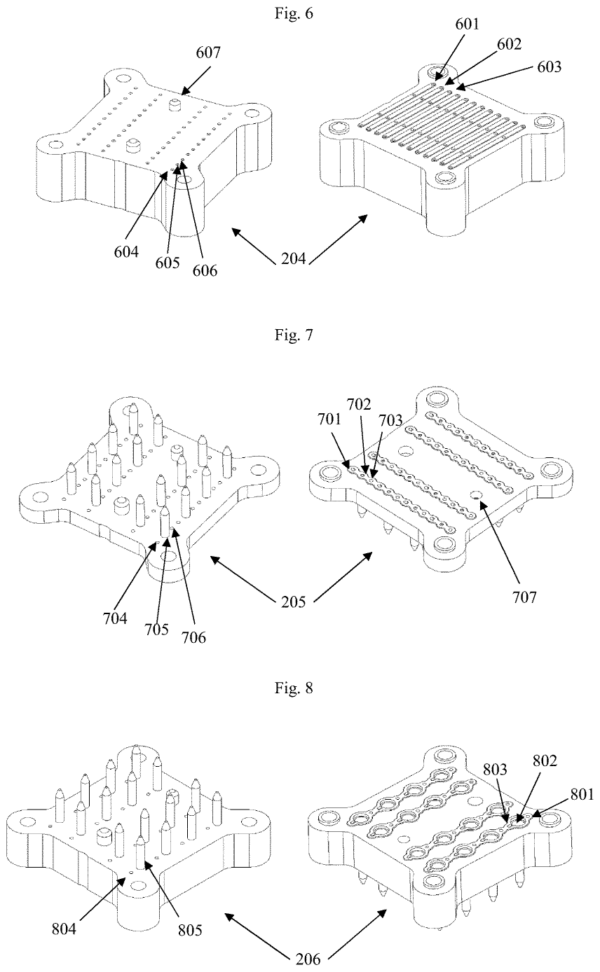

Next, a restriction module is present. FIG. 6 shows a restriction module 204. The restriction module contains the same number of through holes as are necessary to manufacture all the fibres, in this case three fluids per fibre (cross-linking solution, fluid A and fluid B) times sixteen fibres, so, 48 channels. On the bottom base of the restriction module, similarly to the multiplying module, a blind or closure for the channels of the previous module is present, except in select orifices. Each of the linear blinds, solid areas or closures that can be seen on the base of restriction module 204 aligns with a channel from the top base of multiplying module 203. Four orifices are present on each blind. Taking only the orifices that will make up a single fibre, the relevant connections are: orifices 601, 602 and 603 respectively receive fluids from channels 504, 505 and 506 that flow through the restriction module inside respective through holes and exit respectively on orifices 604, 605 and 606. Thus, orifice 604 corresponds to the cross linking solution, orifice 605 to fluid A and orifice 606 to fluid B. This pattern repeats along the blind 4 times for each fluid type, and across the remaining blinds another 4 times, forming 16 combinations of channels for 3 fluids. Pin 607 depicts an optional alignment pin that may be used for improved alignment of the modules. The dimensions of a channel dependent on its a diameter/length ratio. Specifically, the channels should be dimensioned such that the flows remains relatively laminar. In practice, this may mean that a channel diameter is first determined, depending on the preferred diameter of the to-be produced co-axially layered hydrogel fiber. Once this is known, a CFD simulation may be performed to determine a range of lengths of the channel such that the flow stays relatively linear. For example, this may mean that the diameter of a channel is in an order of about 0.5 cm and having a length of about 2 cm.

The following module is a first fabrication module, such as the one, 205, depicted on FIG. 7. Each of the orifices on the top base of the restriction module now have a corresponding orifice on the bottom base of the first fabrication module. Orifice 604 corresponds to orifice 701, 605 to 702 and 606 to 703. Orifice 701 feeds orifice 704, orifice 705 fees nozzle 705 and orifice 703 feeds orifice 706. Orifice 704 thus transports the cross-linking solution and orifice 706 transports fluid B. The nozzle 705 receives fluid A and has an opening on the top to form the first layer, or core, of the fibre, in a liquid state. The module's base presents with an optional pinhole 707 to receive an optional alignment pin on the top base a previous module.

A second fabrication module 206 is then provided, an example of which is shown in FIG. 8. This second fabrication module is configured so the nozzle 705 (and its similar nozzles on the first fabrication module) of the first fabrication module is able to penetrate it in orifice 802. Orifice 802 is configured to be concentric with nozzle 705 and leave a free space, generally cylindrically shaped, preferably tapering at the top, between its inner surface and the outer surface of nozzle 705, forming a further nozzle, 805. Orifice 801 receives the cross-linking solution which flow through to orifice 804. The small side notch 803 visible next to orifice 802 in FIG. 8 serves to flood the free space between nozzle 705 and orifice 802/nozzle 805 with fluid B, allowing for the second layer, or the sheath, to form co-axially to the first layer, both layers exiting nozzle 805 through a top hole. Due to the combination of the overall dimensions of the channels, flow rates and pressures of both fluids, the inventors were able to provide for a non-turbulent laminar flow at the outlet of nozzle 805, meaning that fluids A and B remain separated in a co-axial fashion.

Finally, a cross-linking module is provided, an example of which can be seen in FIG. 9. This cross-linking module is configured so the nozzle 805 (and its similar nozzles on the second fabrication module) of the second fabrication module is able to penetrate it in orifice 901. Orifice 901 is configured to be concentric with nozzle 805 and leave a free space, generally cylindrically shaped, tapering at the top, between its inner surface and the outer surface of nozzle 805, forming a further nozzle, 903. The dimension of this free space may be similar to the dimensions of the free space in the previous module. The small side notch 902 visible next to orifice 901 in FIG. 9 serves to flood the free space between nozzle 805 and orifice 901/nozzle 903 with cross-linking solution, allowing for the cross-linking solution to envelope, co-axially to the first and second layers, the joint precursor fluid flow. Cross-linking starts immediately on contact of the cross-linking solution with the second layer and the cross-linking ions quickly diffuse through the newly formed hydrogel network due to, by definition of an hydrogel, the mass of the network being mostly water, and also cross-link the first layer (when this one is made up from a cross-linkable hydrogel material).

The hydrogel fibre and leftover cross-linking solution then exit through the top orifice of nozzle 903. Nozzle 903 may then be connected to any kind of transport system, such as hoses, or eject into a vessel, or, as will be shown in another embodiment, feed the fibres into a set of merging modules.

In the particular embodiment that has just been described, the cross-linking module fabricates sixteen double layered hydrogel fibres. It is clear that the modules may be configured, under the general principles describe above, to fabricate any number of fibres with any number of layers.

A Flowcell with a Merging Section

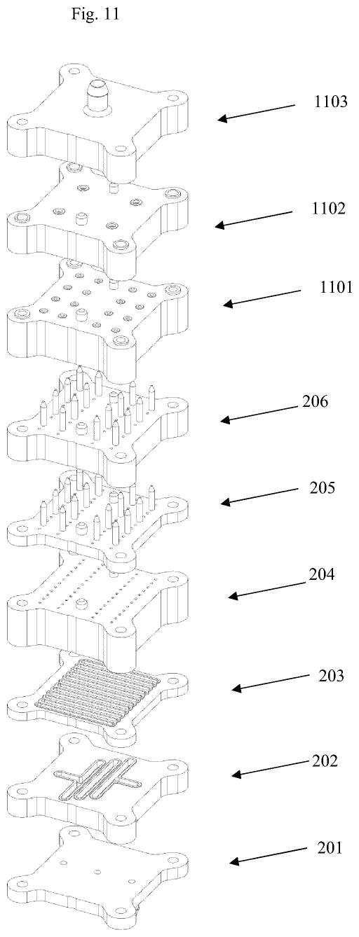

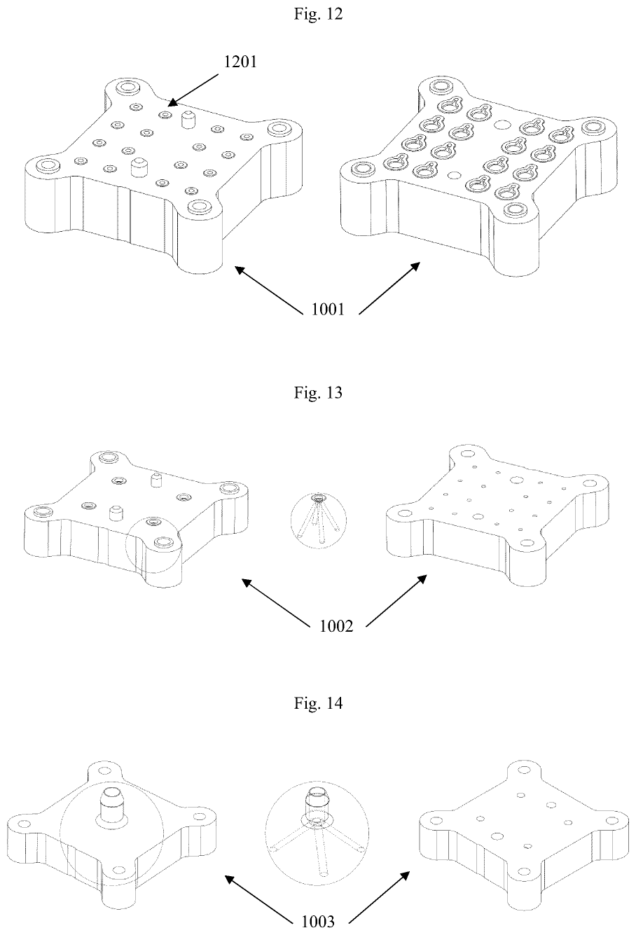

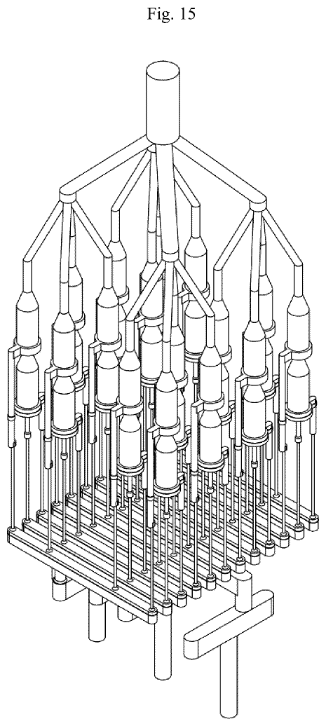

A further embodiment of the invention is one in which the multiple fabricated (i.e. cross-linked) hydrogel fibres are merged into a single output from the flowcell. An example will be described by starting from the 16 fibres flowcell of the previous embodiment. FIG. 11 shows an exploded view of a flowcell according to this embodiment. The flowcell is the same as for the precious embodiment up to the second fabrication module 206. Cross linking module 207 is replaced by a different cross-linking module 1101 that allows the hydrogel fibres to feed into a first merging module 1102. The only difference between these cross-linking modules is that in this embodiment the module has no top nozzles 903, ending on a flat face with orifices. The nozzles 805 of the second fabrication module still penetrate this crosslinking module and the fibres are still cross-linked. After cross-linking the fibres and the leftover cross-linking fluid flow into the first module of the merging section, merging module 1002. FIG. 13 shows a merging module according to this embodiment of the invention. The sixteen orifices 1201 of the cross-linking module 1101 feed into sixteen orifices on the base of module 1002. In this particular configuration, sixteen is dividable by four so the first merging module is configured to merge the flow of four base orifices into a single top orifice. The middle part of FIG. 13 depicts the interior of module 1002 at the circled section on the left top isometric view. In this middle part a possible configuration for joining the flows of four orifices into one is seen, with four diagonal channels starting at the bottom and merging at the top to combine the flows. At the top base of the first merging module, each orifice now outputs four hydrogel fibres.

A second merging module 1003 presents with four orifices on the bottom base that respectively receive the four hydrogel fibres and any leftover cross-linking fluid. Similarly to the first merging module, these four bottom base orifices are connected to a single top orifice or nozzle, by internal merging channels, for example diagonal in nature as seen in the central part of FIG. 14, that, similarly to FIG. 13, shows the interior of the module near the circled zone on the left top isometric view. The top orifice or nozzle outputs the sixteen hydrogel fibres at the same time.

The top orifice or nozzle of the second merging module can now be connected to any kind of transport system, such as hoses, or eject into a vessel.

As before, the particular merging section described corresponds to a sixteen fibres flowcell, but depending on the number of these either merging modules with more or less orifices, or using more or less merging modules may be appropriate. For example, a single merging module may sometimes be enough. It may also be desirable that the final merging module outputs the fibres at more than a single nozzle. For example, the sixteen fibres of the previous embodiment may be output by two nozzles on a single merging section, with eight fibres per nozzle, utilising a single merging module.

1. Modular flow cell for production of co-axially layered hydrogel fibers, comprising a plurality of sections, wherein each of the plurality of sections is in fluid communication with an adjacent section of the plurality of sections, and wherein the plurality of sections comprises:
an inlet section arranged to receive at least a first material, a second material and a crosslinking solution via corresponding connectors;
a first fabrication section arranged to receive the first material from the inlet section and feed it to a second fabrication section via at least two main channels, forming a first layer fluid flow, and receive the second material and crosslinking solution from the inlet section and feed it to a second fabrication section via individual first fabrication section channels;
wherein the second fabrication section is arranged to receive the first layer fluid flow and the second material from the first fabrication section, and feed the second material concentrically around the first layer fluid flow, thereby forming a co-axial fluid flow, and wherein the second fabrication section is further arranged to feed the co-axial fluid flow to a cross linking section via the at least two main channels and the crosslinking solution via corresponding second fabrication section channels to a cross-linking section;
wherein the cross-linking section is arranged to receive the co-axial fluid flow and crosslinking solution from the second fabrication section, and feed the crosslinking solution concentrically around the co-axial fluid, thereby forming a co-axially layered hydrogel fiber in the at least two main channels;
a distribution section provided upstream of the first fabrication section arranged to direct the at least first material, second material and crosslinking solution from an upstream section into at least three distribution section channels, said distribution section channels having a geometry adapted to be connected to a downstream section and are arranged to keep the first material, second material and crosslinking solution separated
and
wherein the modular flow cell comprises at least one module, wherein the at least one module comprises at least one section of the plurality of sections.

2. (canceled)

3. Modular flow cell according to claim 12, wherein the plurality of sections further comprises a multiplying section provided downstream of the distribution section, wherein the multiplying section is arranged to receive each of the first material, second material and crosslinking solution from the distribution section channels in at least one individual multiplying section channel, wherein each of the at least one individual multiplying section channels diverts in to a plurality of multiplying section subchannels arranged to feed the first material, second material and crosslinking solution to a further downstream section.

4. Modular flow cell according to claim 1, wherein the plurality of sections further comprises a restriction section provided upstream of the first fabrication section arranged to substantially equalize the pressure in the first fabrication section channels via restriction channels, wherein the restriction channels have a cross-section that is smaller than the upstream channels.

5. Modular flow cell according to claim 1, wherein the plurality of sections further comprises a merging section provided downstream of the cross-linking section arranged to merge multiple co-axially layered hydrogel fibers, wherein the merging section comprises a plurality of merging section subchannels arranged to receive a single upstream formed co-axially layered hydrogel fiber per subchannel and wherein the multiple subchannels merge in to a merging section channel downstream of the merging section subchannels.

6. Modular flow cell according to claim 1, wherein the co-axially layered hydrogel fibers are appropriate for a cell culture, preferably appropriate for cultured meat applications.

7. Modular flow cell according to claim 1, wherein the at least one module is provided by a plurality of modules, wherein each module of the plurality of modules is configured for being fixed to at least one further module of the plurality of modules, for enabling the plurality of modules to jointly provide the plurality of sections.

8. Modular flow cell according to claim 7, wherein the plurality of modules comprise at least one element of a group comprising: an inlet module for providing at least the inlet section, a first fabrication module for providing at least the first fabrication section, a second fabrication module for providing at least the second fabrication section, a cross-linking module for providing at least the cross-linking section.

9. Modular flow cell according to claim 7, wherein the plurality of sections comprise at least one third fabrication section, wherein the at least one third fabrication section is configured for receiving the co-axial fluid flow and at least one further second material from an upstream module, for feeding the further second material concentrically around the co-axial fluid flow for adding an additional co-axial layer thereto, and for feeding the co-axial fluid flow to a downstream module via the at least two main channels.

10. Modular flow cell according to claim 1, wherein the at least two main channels are provided by a plurality of N main channels for the simultaneous production of N co-axially layered hydrogel microfibers in an industrial production arrangement.

11. (canceled)

12. (canceled)

13. (canceled)

14. Module for use in a modular flow cell according to claim 1.

15. Module according to claim 14, wherein the module is at least one element of a group comprising: an inlet module for providing at least the inlet section, a first fabrication module for providing at least the first fabrication section, a second fabrication module for providing at least the second fabrication section, a cross-linking module for providing at least the cross-linking section, a distribution module for providing at least the distribution section.
